(19) United States
(12) Patent Application Publication
(10) Pub. No.: US 2018/0187315 A1
(43) Pub. Date: Jul. 5, 2018

(54) PROCESS FOR DEGREASING A CHEMICAL PLANT

(71) Applicant: SHELL OIL COMPANY, Houston, TX (US)

(72) Inventors: Johannes Leendert Willem Cornelis DEN BOESTERT, Amsterdam (NL); Sanne WIJNANS, Amsterdam (NL)

(21) Appl. No.: 15/740,013

(22) PCT Filed: Jun. 28, 2016
(86) PCT No.: PCT/EP2016/064923

§ 371 Date: Dec. 27, 2017

(30) Foreign Application Priority Data
Jun. 30, 2015 (EP) 15174681.5

(51) Int. Cl. C23G 5/032; C11D 11/00; C11D 7/50; C07C 68/06

(52) U.S. Cl. CPC C23G 5/032; C11D 11/0041; C07C 69/96; C07C 68/06; C11D 7/5022

(57) ABSTRACT

A process for degreasing a chemical plant is disclosed. The vessels and process lines are rinsed with a solvent comprising at least 50 wt % dialkyl carbonate, based upon the weight of the solvent.

### FIELD OF THE INVENTION

The present invention relates to a process for degreasing a chemical plant.

### BACKGROUND OF THE INVENTION

Chemical plants typically consist of vessels such as reactors, storage vessels and distillation columns that are connected by process lines. The vessels and process lines are usually made of stainless steel or carbon steel. Before start-up or after a turnaround it is usually necessary to clean the vessels and process lines because the internal surfaces may have become contaminated with greasy substances. Cleaning and degreasing agents are commercially available and can be used to clean the chemical plant. However, these agents may leave cleaning residues on the metal surfaces and elaborate water rinsing and drying procedures have to be used to ensure that the chemical plant is ready for use.

The present inventors have sought to provide an alternative method for removing greasy substances from the internal surfaces of vessels and process lines in a chemical plant.

### SUMMARY OF THE INVENTION

Accordingly, the present invention provides a process for degreasing a chemical plant comprising vessels and process lines connecting the vessels, comprising a step of rinsing the vessels and process lines with a solvent, wherein the solvent comprises at least 50 wt % dialkyl carbonate, based upon the weight of the solvent.

The present inventors have found that this process can provide effective degreasing of a chemical plant and there is no need for additional steps of water rinsing and drying to remove residues from the metal surfaces.

### DETAILED DESCRIPTION OF THE INVENTION

The vessels in the chemical plant suitably include storage tanks, distillation columns, accumulator vessels and reactors. The vessels and process lines connecting those vessels are suitably made from metal and preferably are made from steel, e.g. stainless steel or carbon steel.

The vessels and process lines are rinsed with a solvent. Preferably the solvent is flushed through the vessels and process lines for at least 6 hours, preferably at least 12 hours and more preferably at least 24 hours. The solvent is “flushed through” by continually recirculating the solvent through the chemical plant. The solvent is preferably at ambient temperature but may also be heated. In a preferred embodiment of the invention the vessels include at least one distillation column and the solvent is heated in the distillation column such that the column is in a state of reflux. This enables the solvent to reach all the surfaces within the distillation column, thereby providing effective degreasing. The amount of solvent that is required is suitably determined by the volume of the chemical plant.

The solvent comprises at least 50 wt % dialkyl carbonate, based upon the weight of the solvent, preferably comprises at least 70 wt % dialkyl carbonate, more preferably comprises at least 90 wt % dialkyl carbonate and most preferably is pure dialkyl carbonate. The dialkyl carbonate suitably has C₁₋₄ alkyl groups, and is preferably dimethyl carbonate or diethyl carbonate and is most preferably diethyl carbonate.

After use the solvent may be treated as waste or may be cleaned up and re-used. If pure dialkyl carbonate is used as the solvent it may be distilled after use and the resulting pure dialkyl carbonate can be used in the process of the invention or can be used as a feedstock in a process for preparing diaryl carbonate.

In a preferred embodiment of the invention, the chemical plant is a plant for preparing diaryl carbonate. Preferably the diaryl carbonate is prepared from a dialkyl carbonate and an aryl alcohol. Preferably the dialkyl carbonate in the solvent is the same dialkyl carbonate as used in the process for preparing the diaryl carbonate. Using the same dialkyl carbonate is advantageous because the presence of residual dialkyl carbonate after the degreasing process has no disadvantageous effect on the process for preparing diaryl carbonate. Using pure dialkyl carbonate is particularly advantageous because there are no residual components in the chemical plant that may detrimentally affect the preparation of the diaryl carbonate.

The invention further provides a process for preparing diaryl carbonate from a dialkyl carbonate and an aryl alcohol in a chemical plant comprising vessels and process lines connecting the vessels, comprising a step of rinsing the vessels and process lines with a solvent, wherein the solvent comprises at least 50 wt % dialkyl carbonate, based upon the weight of the solvent, wherein this step is carried out prior to reaction of the dialkyl carbonate and the aryl alcohol. Preferably the solvent is pure dialkyl carbonate and is the same as the dialkyl carbonate used to prepare the diaryl carbonate.

In a most preferred embodiment of the invention, the chemical plant is a plant for preparing diphenyl carbonate. Preferably the diphenyl carbonate is prepared from diethyl carbonate and phenol. Alternatively the diphenyl carbonate may be prepared from dimethyl carbonate and phenol. Reaction preferably takes place in the presence of a transesterification catalyst. Suitable catalysts and reaction conditions are known to the skilled person and are described, for example, in U.S. Pat. No. 5,344,954. Preferably the dialkyl carbonate in the solvent is diethyl carbonate and preferably the solvent is pure diethyl carbonate. In an alternative embodiment the dialkyl carbonate in the solvent is dimethyl carbonate and preferably the solvent is pure dimethyl carbonate. Rinsing the vessels and process lines with diethyl carbonate or dimethyl carbonate provides effective degreasing and any residual diethyl carbonate or dimethyl carbonate does not need to be removed before starting or restarting the process for preparing diphenyl carbonate.

The invention further provides a process for preparing diphenyl carbonate from diethyl carbonate and phenol in a chemical plant comprising vessels and process lines connecting the vessels, comprising a step of rinsing the vessels and process lines with a solvent, wherein the solvent comprises at least 50 wt % dialkyl carbonate, based upon the weight of the solvent, wherein this step is carried out prior to reaction of the diethyl carbonate and the phenol. Preferably the solvent is pure diethyl carbonate.

The invention yet further provides a process for preparing diphenyl carbonate from dimethyl carbonate and phenol in a chemical plant comprising vessels and process lines connecting the vessels, comprising a step of rinsing the vessels and process lines with a solvent, wherein the solvent comprises at least 50 wt % dialkyl carbonate, based upon the weight of the solvent, wherein this step is carried out prior to reaction of the dimethyl carbonate and the phenol. Preferably the solvent is pure dimethyl carbonate.

The invention is further illustrated by means of the following experiments.

### EXAMPLES

Steel strips were partially immersed in an oil mixture and then left to dry in the air. The strips of the examples and comparative example (but not the control) were then placed in tubes filled with 10 ml of room temperature solvent and placed in an ultrasonic bath for approximately 5 minutes. Table 1 summarises the different strips, oil/water mixtures and solvents that were used:

TABLE 1

| | Steel | Oil mixture | Solvent |
|---|---|---|---|
| Control | 316 | Ecoolant | None |
| | stainless | Clear M¹ | |
| | steel | | |
| Comparative | 316 | Ecoolant | Monoethylene |
| Example 1 | stainless | Clear M¹ | glycol² |
| | steel | | |
| Example 1 | 316 | Ecoolant | Diethyl |
| | stainless | Clear M¹ | carbonate³ |
| | steel | | |
| Example 2 | 316 | RTD Liquid⁴ | Diethyl |
| | stainless | | carbonate³ |
| | steel | | |
| Example 3 | 316 | Copaslip⁵ | Diethyl |
| | stainless | | carbonate³ |
| | steel | | |
| Example 4 | Carbon steel | Ecoolant | Diethyl |
| | | Clear M¹ | carbonate³ |

¹Ecoolant Clear M is a 7-10% stabilized solution of vegetable oils in water. It is used as a cooling agent for metal machining purposes and marketed by Eco-Point.
²Monoethylene glycol was 99.5% Ensure grade from Merck.
³Diethyl carbonate was synthesis grade from Merck.
⁴RTD Liquid is a reaming, tapping and drilling lubricant marketed by Rocol. This oil consists of 70% to 90% chlorinated C₁₄-C₁₇ paraffins with, given the reported density of 1.2 g/ml, an assumed chlorine concentration above 40 wt %.
⁵Copaslip is a heavy duty anti-seize compound marketed by Molyslip. It is a paste of micro sized copper particles, oxidation and corrosion inhibitors and a bentonite clay thickener in heavy paraffinic base oil.

The steel strips were visually inspected. For comparative example 1 and example 1 a thin paper napkin was rubbed over the area that was submersed into the solvent to assess how much of the oil film remained on the metal surface. The results are summarised in table 2 below:

TABLE 2

| | Results |
|---|---|
| Control | Oil stain visible after rubbing paper |
| | napkin on the strip. |
| Comparative | Visible removal of vegetable oil film. |
| Example 1 | No oil stain on paper napkin. |
| Example 1 | Visible removal of vegetable oil film. |
| | No oil stain on paper napkin. |
| Example 2 | Visible removal of paraffinic oil |
| | film. |
| Example 3 | Visible removal of paraffinic oil |
| | film. |
| Example 4 | Visible removal of vegetable oil film. |

The diethyl carbonate was capable of removing a variety of oily residues from both stainless steel and carbon steel and provided similar degreasing to monoethylene glycol.

1. A process for degreasing a chemical plant comprising vessels and process lines connecting the vessels, the process comprising a step of rinsing the vessels and process lines with a solvent, wherein the solvent comprises at least 50 wt % dialkyl carbonate, based upon the weight of the solvent.

2. The process according to claim 1, wherein the solvent comprises at least 90 wt % dialkyl carbonate.

3. The process according to claim 1, wherein the solvent is pure dialkyl carbonate.

4. The process according to claim 1, wherein the dialkyl carbonate is dimethyl carbonate or diethyl carbonate.

5. The process according to claim 1, wherein the chemical plant is a plant for preparing diaryl carbonate from a dialkyl carbonate and an aryl alcohol.

6. The process according to claim 5, wherein the dialkyl carbonate in the solvent is the same dialkyl carbonate as used in the process for preparing the diaryl carbonate.

7. The process according to claim 1, wherein the chemical plant is a plant for preparing diphenyl carbonate from diethyl carbonate and phenol or from dimethyl carbonate and phenol.

8. The process according to claim 7, wherein the solvent is pure diethyl carbonate or pure dimethyl carbonate.

9. A process for preparing diaryl carbonate from a dialkyl carbonate and an aryl alcohol in a chemical plant comprising vessels and process lines connecting the vessels, comprising a step of rinsing the vessels and process lines with a solvent, wherein the solvent comprises at least 50 wt % dialkyl carbonate, based upon the weight of the solvent, wherein this step is carried out prior to reaction of the dialkyl carbonate and the aryl alcohol.

10. A process for preparing diphenyl carbonate from diethyl carbonate and phenol in a chemical plant comprising vessels and process lines connecting the vessels, comprising a step of rinsing the vessels and process lines with a solvent, wherein the solvent comprises at least 50 wt % dialkyl carbonate, based upon the weight of the solvent, wherein this step is carried out prior to reaction of the diethyl carbonate and the phenol.

11. The process according to claim 10, wherein the solvent is pure diethyl carbonate.

12. A process for preparing diphenyl carbonate from dimethyl carbonate and phenol in a chemical plant comprising vessels and process lines connecting the vessels, comprising a step of rinsing the vessels and process lines with a solvent, wherein the solvent comprises at least 50 wt % dialkyl carbonate, based upon the weight of the solvent, wherein this step is carried out prior to reaction of the dimethyl carbonate and the phenol.

13. The process according to claim 12, wherein the solvent is pure dimethyl carbonate.
